SEQUENCE LISTING (14 sequences)

SEQ ID NO: 1 - DNA, 1050 bp, Aeropyrum pernix K1
gtgatggtcgcgtcgaccttcgtagtagtgttccagggcttcggcctgacagcgccgcagggaggcggctccagcccctccggcggcggggaggagggagggctggaggagccgcagggcctcttccccacggtatcatataccctacccctcgcaggaggggataggcttgtatacgagtccacctccacgtcagcgagcggcaccaacgtggccacaaacgcggtctccatagtcgagccaggctggcccgagagcagtgttgaggtccagctcctggaggccggagacccggttgtaacgtcgacgccggagaagggtgtgctcaccacaacccttctagccctgccgaaggagtatctgggtatgcaggagattgtgatacccgtgtacataccgcccggccgctcaggcctctgcatgaggctaacgctagaagccggggaggcaggcgggtacacatacagaggctacgcaaacgtgggggactacactatagctgtcaaagcggtctacaggggcgacgggatacttgaggcctttgaagccggcatagtcgggggcggcctatccatacgctacacccagagcctggtggaggctagcgtctccggctccgacacgatggttagcgtggagtgggagtgcaccgcggacgggttcagcagcaacctaagctacgtgaaggagggtctcgccgtcctggaggacgggaggctaatatacataacccccgaggagttcaggcagctgctccagggcgacgctatactggcggtctacagcaaaacctgcccccactgccacagggactggccacagctgatccaggcctcgaaggaggtggatgtgcccatagtcatgttcatatggggcagcctcataggggagagggagctctccgccgccaggctcgagatgaacaaggccggtgtggagggcacgccaaccctagtgttctacaaggaagggaggatagtggacaagctggtgggcgcaacgccctggagcctcaaggtggagaaggctagggagatatacgggggctga

SEQ ID NO: 2 - protein, 349 aa, Aeropyrum pernix K1
VMVASTFVVVFQGFGLTAPQGGGSSPSGGGEEGGLEEPQGLFPTVSYTLPLAGGDRLVYESTSTSASGTNVATNAVSIVEPGWPESSVEVQLLEAGDPVVTSTPEKGVLTTTLLALPKEYLGMQEIVIPVYIPPGRSGLCMRLTLEAGEAGGYTYRGYANVGDYTIAVKAVYRGDGILEAFEAGIVGGGLSIRYTQSLVEASVSGSDTMVSVEWECTADGFSSNLSYVKEGLAVLEDGRLIYITPEEFRQLLQGDAILAVYSKTCPHCHRDWPQLIQASKEVDVPIVMFIWGSLIGERELSAARLEMNKAGVEGTPTLVFYKEGRIVDKLVGATPWSLKVEKAREIYGG

SEQ ID NO: 3 - DNA, 1032 bp, Aeropyrum pernix K1
gtgattaggtgcgtgattatgccgctcaggctctctgcggtgagggcgcctaagataccccgtggggaggagtacgacaccgtcatagtgggggcggggcctgcgggcctctcggcagccatatacacaacaaggttcctcatgtcgacactcatagtctcgatggacgtgggtggacagctaaacctcaccaactggatagacgactaccccggcatgggtgggctggaggcgtcgaagctcgtggagagcttcaagagccacgcagaaatgttcggcgccaagatagtgactggggtgcaggtcaagactgttgacaggctcgacgacggctggttcctagtgagggggtccagggggctggaggtgaaggcccgcaccgtcatactggcggtggggagcaggaggaggaaactcggcgtccccggggaggcggagctcgcgggcaggggcgtcagctactgcagcgtgtgcgacgcgcccctgttcaagggtaaggacgccgtggttgttgtggggggcggcgactccgccctcgagggggccctcctcctcagcggctacgtcgggaaggtctacctggtccacaggaggcaggggttcagggcgaagcccttctacgtggaggaggcgaggaagaagcctaacattgagttcatcctagacagcatagtgaccgagataagagggcgggaccgggtggagtctgtggtcgtgaagaacaaggtgaccggcgaggagaaggagctcagggtggacgggatcttcatagagataggctccgagccgccgaaggagctgttcgaggccatagggctggagaccgatagcatgggcaacgtggtggttgacgagtggatgaggacgagcatcccagggatattcgcggcgggagactgcaccagcatgtggccgggcttcaggcaggtggtcaccgccgcggcgatgggcgcggtggccgcctacagcgcctacacctacctgcaggagaagggcctctacaagccgaagcctttaactgggttaaagtaa

SEQ ID NO: 4 - protein, 343 aa, Aeropyrum pernix K1
VIRCVIMPLRLSAVRAPKIPRGEEYDTVIVGAGPAGLSAAIYTTRFLMSTLIVSMDVGGQLNLTNWIDDYPGMGGLEASKLVESFKSHAEMFGAKIVTGVQVKTVDRLDDGWFLVRGSRGLEVKARTVILAVGSRRRKLGVPGEAELAGRGVSYCSVCDAPLFKGKDAVVVVGGGDSALEGALLLSGYVGKVYLVHRRQGFRAKPFYVEEARKKPNIEFILDSIVTEIRGRDRVESVVVKNKVTGEEKELRVDGIFIEIGSEPPKELFEAIGLETDSMGNVVVDEWMRTSIPGIFAAGDCTSMWPGFRQVVTAAAMGAVAAYSAYTYLQEKGLYKPKPLTGLK

SEQ ID NO: 5 - DNA, 753 bp, Aeropyrum pernix K1
atgcccgggagcatacccctgatcggagagagattccctgaaatggaggttactacagaccacggtgtaatcaagctaccagaccactatgtgagccagggtaagtggttcgtgctgttcagccatccagcagatttcactcccgtctgcacgacagagttcgtcagctttgctaggagatacgaggacttccagaggcttggagtcgacctgataggtctcagcgttgacagtgtgttcagccacataaagtggaaggagtggattgagaggcacattggcgttaggataccgttcccgataatagcggatccgcagggaactgtggctaggaggctgggtctacttcacgccgagagcgccacacacacggttagaggggtattcatagtcgatgctaggggcgttatcaggactatgctctactaccccatggagcttggcagacttgtagacgagatactgaggatagttaaggccctgaagctaggcgacagcctgaagagggcagtacccgcagactggcccaacaacgagataattggtgagggactcatagttccgccgccaactacggaggaccaggcgagggcgaggatggagtcgggccagtaccgctgtctagactggtggttctgctgggacactccagcaagcagggacgacgtggaggaggctaggagatacctcagaagggccgctgagaagcccgctaagctgctctatgaggaagcccgaacacacctacactag

SEQ ID NO: 6 - protein, 250 aa, Aeropyrum pernix K1
MPGSIPLIGERFPEMEVTTDHGVIKLPDHYVSQGKWFVLFSHPADFTPVCTTEFVSFARRYEDFQRLGVDLIGLSVDSVFSHIKWKEWIERHIGVRIPFPIIADPQGTVARRLGLLHAESATHTVRGVFIVDARGVIRTMLYYPMELGRLVDEILRIVKALKLGDSLKRAVPADWPNNEIIGEGLIVPPPTTEDQARARMESGQYRCLDWWFCWDTPASRDDVEEARRYLRRAAEKPAKLLYEEARTHLH

SEQ ID NO: 7 - DNA, 681 bp, Pyrococcus horikoshii OT3
atgggactaataagtgaggaggacaagaggataattaaggaagagttcttctcaaagatggtgaacccagtcaagctcatcgtcttcataggaaaagaacactgccaatactgtgatcagcttaagcaattagttcaggagctctcagagctgacagataagctgagctatgagatagttgacttcgacactcccgagggaaaggagctagctgagaagtacaggatcgacagggccccagcaactacaataacccaggatggaaaggacttcggcgttagatacttcggaattccagctggacacgagtttgcagcatttcttgaggatatagttgatgtaagcaagggagacaccgatttaatgcaggatagcaaggaggaggtttcaaagatagacaaagacgtcaggatattgatcttcgtaacgccaacctgcccatactgtccattagccgttagaatggcccacaagttcgcaatcgagaacacaaaagctggaaaaggaaagatccttggagacatggtggaagctatagagtatccagaatgggccgatcagtacaacgtcatggccgttccaaagatagtaatacaggtaaatggagaggataaagtccaattcgagggggcttacccagagaaaatgttcctggaaaagctactttcagccctaagctag

SEQ ID NO: 8 - protein, 226 aa, Pyrococcus horikoshii OT3
MGLISEEDKRIIKEEFFSKMVNPVKLIVFIGKEHCQYCDQLKQLVQELSELTDKLSYEIVDFDTPEGKELAEKYRIDRAPATTITQDGKDFGVRYFGIPAGHEFAAFLEDIVDVSKGDTDLMQDSKEEVSKIDKDVRILIFVTPTCPYCPLAVRMAHKFAIENTKAGKGKILGDMVEAIEYPEWADQYNVMAVPKIVIQVNGEDKVQFEGAYPEKMFLEKLLSALS

SEQ ID NO: 9 - DNA, 1011 bp, Pyrococcus horikoshii OT3
gtggaggtgaaggaaatgttcagcctaggtgggggtttaggtaggagtaaggttgatgagagcaaggtctgggatgttataatcataggagcagggcccgcgggatacacagcagcaatctacgctgcgagattcggattagacactataattattacaaaggatctaggaggaaacatggcaattacggatctaatagaaaactatcctggattccccgagggtataagtggttccgaactatcgaagaagatgtatgatcaagttaagaagtatggtgtcgaagtaataattgatgaagtcatccgcatagatccagctgagtgtgcttactatgaagggccctgtaattttgtagtcaaaactgctaatggaaaagaatacaaagcaaaaactataataattgccgttggtgcagaaccaagaaaactcaatgttccaggggagaaggaatttactggaagaggtgttagctactgtgctacttgtgatggaccattattcgtaggaaaggaagtcatagttgttggtggtggaaatacagcgttacaggaagctttataccttcacagcataggtgtcaaggtaaccctagttcacagaagggataaatttagagctgacaagatacttcaggataggtttaagcaggcgggaatccctgctatcctgaatacagtcgttaccgaaattaaggggactaacaaagttgagagtgttgttcttaagaacgttaagacgggagaaacggttgagaagaaggtcgatggtgtcttcatattcataggttacgagcctaagacggacttcgttaagcatttggggataacagatgaatatggttacattccagttgatatgtacatgagaactaaggttccaggaatattcgctgcaggagacataactaacgtgttcaagcagattgccgtcgcagtgggtcagggagcaattgcagcaaactctgctaaggagtttatagaaagctggaatggaaagactattgagtga

SEQ ID NO: 10 - protein, 336 aa, Pyrococcus horikoshii OT3
VEVKEMFSLGGGLGRSKVDESKVWDVIIIGAGPAGYTAAIYAARFGLDTIIITKDLGGNMAITDLIENYPGFPEGISGSELSKKMYDQVKKYGVEVIIDEVIRIDPAECAYYEGPCNFVVKTANGKEYKAKTIIIAVGAEPRKLNVPGEKEFTGRGVSYCATCDGPLFVGKEVIVVGGGNTALQEALYLHSIGVKVTLVHRRDKFRADKILQDRFKQAGIPAILNTVVTEIKGTNKVESVVLKNVKTGETVEKKVDGVFIFIGYEPKTDFVKHLGITDEYGYIPVDMYMRTKVPGIFAAGDITNVFKQIAVAVGQGAIAANSAKEFIESWNGKTIE

SEQ ID NO: 11 - DNA, 33 bp, Artificial Sequence
ggaattccatatgggactaataagtgaggagga

SEQ ID NO: 12 - DNA, 29 bp, Artificial Sequence
cgggatcctagcttagggctgaaagtagg

SEQ ID NO: 13 - DNA, 32 bp, Artificial Sequence
ggaattccatatggaggtgaaggaaatgttca

SEQ ID NO: 14 - DNA, 31 bp, Artificial Sequence
cgggatcctcactcaatagtctttccattcc

(19) United States
(12) Patent Application Publication
(10) Pub. No.: US 2003/0235902 A1
(43) Pub. Date: Dec. 25, 2003

(54) Heat-resistant thioredoxin and related enzymes

(76) Inventors: Kazuhiko Ishikawa, Ikeda-shi (JP); Sung-Jong Jeon, Ikeda-shi (JP); Yasuhiro Kashima, Ikeda-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/316,233

(22) Filed: Dec. 10, 2002

(30) Foreign Application Priority Data
Dec. 13, 2001 (JP) 2001-379462
Jul. 16, 2002 (JP) 2002-206352

(51) Int. Cl. C12N 1/20

(52) U.S. Cl. 435/252.3

(57) ABSTRACT

The inventors isolated from hyperthermophilic archaebacteria Aeropyrum pernix a heat resistant thioredoxin, thioredoxin reductase and thioredoxin peroxidase having at least 50% residual activity after heat treatment at 100° C. for 0.5 hour, and sequenced the amino acid and base sequences. The inventors also isolated from hyperthermophilic archaebacteria Pyrococcus horikoshii a heat resistant thioredoxin, thioredoxin reductase, and thioredoxin peroxidase showing substantially no decline in activity when heat treated at 100° C. for 0.5 hour, and sequenced the amino acid and base sequences.

### BACKGROUND OF THE INVENTION

1. Field of the Invention

The present invention relates to a thioredoxin, a thioredoxin reductase and a thioredoxin peroxidase which are capable of functioning at high temperatures, DNAs encoding these proteins or enzymes, vectors containing the DNAs, transformants which have been transformed with the vectors, and methods for producing the proteins or enzymes using the transformants.

2. Description of the Related Art

Thioredoxin is an electron transfer protein with a molecular weight of 10,000 to 130,000, ubiquitously present in E. coli, yeasts, and higher plants and animals, etc. Thioredoxin has a site where 2 cysteine residues flank 2 amino acids (-Cys-X-X-Cys-) as its active center, and is considered to regulate in vivo redox states through a reversible dithiol oxidation-reduction reaction (T. C. Laurent, B. C. Moor, & P. Reinchard, J. Biol. Chem., 239, 3436-3444 (1964); A. Holmgren, Ann. Rev. Biochem. 54, 237-271 (1989); A. Holmgren, J. Biol. Chem. 264, 13963-13966 (1989); and B. B. Buchanan, P. Schurmann, P. Decottignies, & R. M. Lozano, Arch. Biochem. Biophys. 314, 257-260 (1994)). Thioredoxin exists either in the oxidized form, where the two active center cysteine residues form a disulfide, or in the reduced form, where the residues exist as thiol groups.

Examples of in vivo functions of thioredoxin include the cleavage of protein intra- or inter-molecular S—S bonds. In this reaction, reduced thioredoxin reduces protein disulfide into dithiol and is concurrently oxidized to oxidized thioredoxin. The resulting oxidized thioredoxin is reduced back to reduced thioredoxin by thioredoxin reductase and NADPH. FIG. 5 illustrates the cleavage reaction of such an intramolecular protein S—S bond by reduced thioredoxin.

Other examples of in vivo functions of thioredoxin include the elimination of peroxides such as hydrogen peroxide, lipoperoxides and the like. In this reaction, reduced thioredoxin reduces hydrogen peroxide (O₂H₂) into water (H₂O) or reduces hydroperoxides (ROOOH) into hydroxides (ROH), and is concurrently oxidized to oxidized thioredoxin. This reaction is carried out by thioredoxin peroxidase. The oxidized thioredoxin is reduced back to reduced thioredoxin by thioredoxin reductase and NADPH. FIG. 6 illustrates the reduction reaction of hydrogen peroxide (O₂H₂) into water (H₂O) by reduced thioredoxin.

Further examples of in vivo thioredoxin functions include the prevention of cell damage by UV radiation and the control of transcription factors.

The pharmaceutical use of thioredoxin with such functions has been proposed in order to inhibit inhibitor activity by reduction of digestive enzyme inhibitor proteins which are active in the state having cysteins, to detoxify snake venom protein by eliminating the S—S bond between cysteine residues, to prevent skin inflammation caused by UV radiation, and so forth. The addition of thioredoxin to food products has also been proposed in order to eliminate food allergens by eliminating the S—S bonds between the protein cysteine residues, and the use thereof as cosmetics has also been proposed in order to improve skin chapping caused by oxidative stress resulting from dryness or UV rays (Japanese Unexamined Patent Publication 2001-288103, Japanese Unexamined Patent Publication 2001-520027, and Japanese Unexamined Patent Publication 2000-103743).

Solid or semi-solid drugs, food products, cosmetics, and the like are difficult to sterilize by filtration and are therefore usually sterilized by heating. However, hitherto known thioredoxins have low heat resistance. Thus there is the drawback that non-heat resistant thioredoxins when used as drugs or added to foods cannot be sterilized at high temperatures.

In addition, the solubility of a solute in water generally increases with temperature. Accordingly, the use of a heat resistant, i.e., thermostable thioredoxin, thioredoxin reductase, and thioredoxin peroxidase capable of functioning at high temperatures could allow the efficient synthesis of reduced proteins by protein disulfide reduction, and the elimination of active oxygen in hydrogen peroxide or the like through the action of the enzymes on highly concentrated substrate solutions prepared at high temperature.

### SUMMARY OF THE INVENTION

An object of the present invention is to provide a heat resistant thioredoxin, a heat resistant thioredoxin reductase, and a heat resistant thioredoxin peroxidase, DNAs encoding these proteins or enzymes, vectors comprising the DNAs, transformants transformed with the vectors, and methods for efficiently producing the heat resistant thioredoxin, heat resistant thioredoxin reductase, and heat resistant thioredoxin peroxidase using the transformants.

In their extensive research for achieving the above object, the present inventors focused on hyperthermophilic archaea capable of growing at high temperatures in the range of 90 to 100° C. Archaea are organisms belonging to a third group of organisms distinct from eucaryotes and prokaryotes. Archaea are considered to be descended from primeval organisms, and are special organisms which have not evolved or adapted to ordinary temperature environments.

The inventors were the first to isolate such hyperthermophilic archaea-derived thioredoxin, thioredoxin reductase, and thioredoxin peroxidase. The inventors found that these proteins or enzymes are much more heat resistant than hitherto known thioredoxins or the like, are highly stable at room temperature, i.e., ordinary temperature, and exhibit activity even in the presence of organic solvents.

The inventors succeeded in efficiently producing these proteins or enzymes by incorporating DNAs encoding the proteins or enzymes into vectors.

The present invention has been accomplished on the basis of the above findings, and provides the following thermostable thioredoxin, thermostable thioredoxin reductase, and thermostable thioredoxin peroxidase.

1. A heat resistant thioredoxin having at least 50% residual activity after heat treatment at 100° C. for 0.5 hour.

2. A heat resistant thioredoxin according to item 1, derived from hyperthermophilic archaea Aeropyrum pernix.

3. A polypeptide of (1-1) or (1-2) below:

(1-1) a polypeptide comprising the amino acid sequence of SEQ ID NO. 2; and

(1-2) a polypeptide comprising the amino acid sequence of SEQ ID NO. 2 with 1 or more amino acids deleted, substituted, or added, the polypeptide having at least 50% residual thioredoxin activity after heat treatment at 100° C. for 0.5 hour.

4. DNA of (6-1), (6-2) or (6-3) below:

(6-1) DNA comprising the base sequence of SEQ ID NO. 1;

(6-2) DNA comprising DNA which hybridizes under stringent conditions with DNA consisting of the base sequence of SEQ ID NO. 1 and encodes a polypeptide having at least 50% residual thioredoxin activity after heat treatment at 100° C. for 0.5 hour; and

(6-3) DNA encoding the polypeptide according to item 3

5. A vector comprising the DNA according to item 4.

6. A transformant comprising the vector according to item 5.

7. A method for producing a heat resistant thioredoxin, comprising the steps of culturing the transformant of item 6, and collecting the heat resistant thioredoxin from the transformant.

8. A heat resistant thioredoxin according to item 1 whose activity is substantially unimpaired by heat treatment at 100° C. for 0.5 hour.

9. A heat resistant thioredoxin according to item 8, derived from hyperthermophilic archaea Pyrococcus horikoshii.

10. A polypeptide of (2-1) or (2-2) below:

(2-1) a polypeptide comprising the amino acid sequence of SEQ ID NO. 8; and

(2-2) a polypeptide comprising the amino acid sequence of SEQ ID NO. 8 with 1 or more amino acids deleted, substituted, or added, the polypeptide having thioredoxin activity which is substantially unimpaired by heat treatment at 100° C. for 0.5 hour.

11. DNA of (7-1), (7-2) or (7-3) below:

(7-1) DNA comprising the base sequence of SEQ ID NO. 7;

(7-2) DNA comprising DNA which hybridizes under stringent conditions with DNA consisting of the base sequence of SEQ ID NO. 7 and encodes a polypeptide whose thioredoxin activity is substantially unimpaired by heat treatment at 100° C. for 0.5 hour.

(7-3) DNA encoding the polypeptide according to item 10.

12. A vector comprising the DNA of item 11.

13. A transformant comprising the vector of item 12.

14. A method for producing a heat resistant thioredoxin, comprising the steps of culturing the transformant of item 13 and collecting the heat resistant thioredoxin from the transformant.

15. A heat resistant thioredoxin reductase having at least 50% residual activity after heat treatment at 100° C. for 0.5 hour.

16. A heat resistant thioredoxin reductase according to item 15, derived from hyperthermophilic archaea Aeropyrum pernix.

17. A polypeptide of (3-1) or (3-2) below:

(3-1) a polypeptide comprising the amino acid sequence of SEQ ID NO. 4; and

(3-2) a polypeptide comprising the amino acid sequence of SEQ ID NO. 4 with 1 or more amino acids deleted, substituted, or added, the polypeptide having at least 50% residual thioredoxin reductase activity after heat treatment at 100° C. for 0.5 hour.

18. DNA of (8-1), (8-2) or (8-3) below:

(8-1) DNA comprising the base sequence of SEQ ID NO. 3;

(8-2) DNA comprising DNA which hybridizes under stringent conditions with DNA consisting of the base sequence of SEQ ID NO. 3 and encodes a polypeptide having at least 50% residual thioredoxin reductase activity after heat treatment at 100° C. for 0.5 hour; and

(8-3) DNA encoding the polypeptide according to item 17

19. A vector comprising the DNA according to item 18.

20. A transformant comprising the vector according to item 19.

21. A method for producing a heat resistant thioredoxin reductase, comprising the steps of culturing the transformant of item 20, and collecting the heat resistant thioredoxin reductase from the transformant.

22. A heat resistant thioredoxin reductase according to item 15 whose activity is substantially unimpaired by heat treatment at 100° C. for 0.5 hour.

23. A heat resistant thioredoxin reductase according to item 22, derived from hyperthermophilic archaea Pyrococcus horikoshii.

24. A polypeptide of (4-1) or (4-2) below:

(4-1) a polypeptide comprising the amino acid sequence of SEQ ID NO. 10; and

(4-2) a polypeptide comprising the amino acid sequence of SEQ ID NO. 10 with 1 or more amino-acids deleted, substituted, or added, the polypeptide having thioredoxin reductase activity which is substantially unimpaired by heat treatment at 100° C. for 0.5 hour.

25. DNA of (9-1), (9-2) or (9-3) below:

(9-1) DNA comprising the base sequence of SEQ ID NO. 9;

(9-2) DNA comprising DNA which hybridizes under stringent conditions with DNA consisting of the base sequence of SEQ ID NO. 9 and encodes a polypeptide whose thioredoxin reductase activity is substantially unimpaired by heat treatment at 100° C. for 0.5 hour; and

(9-3) DNA encoding the polypeptide according to item 24.

26. A vector comprising the DNA of item 25.

27. A transformant comprising the vector of item 26.

28. A method for producing a heat resistant thioredoxin reductase, comprising the steps of culturing the transformant of item 27 and collecting the heat resistant thioredoxin reductase from the transformant.

29. A heat resistant thioredoxin peroxydase having at least 50% residual activity after heat treatment at 100° C. for 0.5 hour.

30. A heat resistant thioredoxin peroxydase according to item 29, derived from hyperthermophilic archaea Aeropyrum pernix.

31. A polypeptide of (5-1) or (5-2) below:

(5-1) a polypeptide comprising the amino acid sequence of SEQ ID NO. 6; and

(5-2) a polypeptide comprising the amino acid sequence of SEQ ID NO. 6 with 1 or more amino acids deleted, substituted, or added, the polypeptide having at least 50% residual thioredoxin peroxydase activity after heat treatment at 100° C. for 0.5 hour.

32. DNA of (10-1), (10-2) or (10-3) below;

(10-1) DNA comprising the base sequence of SEQ ID NO. 5;

(10-2) DNA comprising DNA which hybridizes under stringent conditions with DNA consisting of the base sequence of SEQ ID NO. 5 and encodes a polypeptide having at least 50% residual thioredoxin peroxydase activity after heat treatment at 100° C. for 0.5 hour; and

(10-3) DNA encoding the polypeptide according to item 31

33. A vector comprising the DNA according to item 32.

34. A transformant comprising the vector according to item 33.

35. A method for producing a heat resistant thioredoxin peroxydase, comprising the steps of culturing the transformant of item 34, and collecting the heat resistant thioredoxin peroxydase from the transformant.

36. A method for purifying a heat resistant protein, comprising a heating step in which a solution of the heat resistant protein to be purified is incubated for 10 to 120 minutes at a temperature such that incubating the protein for 10 to 30 minutes results in at least 60% residual activity and that is at least 10° C. higher than the critical growth temperature of the host producing the protein.

Use and Effects of the Present Invention

The present invention provides a thioredoxin with excellent heat resistance, i.e., thermostability, a thioredoxin reductase with excellent heat resistance and a thioredoxin peroxidase with excellent heat resistance.

i) Drugs, Food Products, Animal Feed, Cosmetic, Detergents

Specifically, the heat resistant thioredoxin of the invention has the function of eliminating active oxygen and the function of reducing oxidized cysteines in protein. Based on these functions, the heat resistant thioredoxin of the invention can be used as pharmaceuticals in applications such as the prevention and treatment of various diseases caused by active oxygen, the inhibition of digestive enzyme inhibitors, the detoxification of snake venom, scorpion venom, and the like through the oxidation, and the treatment and prevention of skin inflammation caused by UV radiation. It can also be used as an antioxidant for pharmaceuticals.

The heat resistant thioredoxin of the invention can also be used as a food additive in applications such as the elimination of food allergens and the prevention of food oxidation.

The heat resistant thioredoxin of the invention can also be used as an animal feed additive in applications such as the prevention of animal diseases through the elimination of active oxygen and the prevention of animal feed oxidation.

The heat resistant thioredoxin of the invention can also be used as a cosmetic in applications such as the improvement of skin chapping caused by oxidative stress resulting from dryness, UV radiation, or the like. It can also be used as an antioxidant for cosmetics.

The heat resistant thioredoxin of the invention can also be used as a detergent component capable of eliminating protein stains through the reduction of oxidized cysteines in protein.

The thioredoxin reductase of the invention can be used as a pharmaceutical in combination with thioredoxin to maintain the thioredoxin in the active reduced form. The thioredoxin peroxidase of the invention can be added along with thioredoxin to prevent oxidation in pharmaceuticals, food products, cosmetics, and the like.

The thioredoxin, thioredoxin reductase and thioredoxin peroxidase of the invention are heat resistant, allowing pharmaceuticals, food products, cosmetics, animal feed, and the like containing these proteins to be sterilized by heating. The thioredoxin, thioredoxin reductase and thioredoxin peroxidase of the invention have excellent stability at room temperature, resulting in long lasting activity and effects over a long period of use. When these proteins are used as detergents, tableware and the like can be washed in hot detergent solution, resulting in improved washing efficiency.

ii) Enzymes and Reagents for Reaction

The use of the thioredoxin and thioredoxin reductase of the invention allows efficient synthesis of reduced protein in highly concentrated substrate solutions prepared at high temperatures, based on the reduction of the protein cystines.

The use of the thioredoxin and thioredoxin peroxidase of the invention allows efficient elimination of active oxygen from hydrogen peroxide or the like in highly concentrated substrate solutions prepared at high temperatures.

The thioredoxin peroxidase of the invention can be used as a sensor which colors upon reaction with peroxides, and can be used as a peroxidase which is bound to antibodies in Western blotting. In these cases, the enzymatic reaction can be carried out at relatively high temperatures, thereby minimizing the effects of protein contaminants and enhancing detection sensitivity.

Because the thioredoxin, thioredoxin reductase, and thioredoxin peroxidase of the invention have excellent stability at room temperature, they can be stored for a relatively long period of time and can withstand repeated use.

Proteins and enzymes generally tend to lose activity in the presence of organic solvents. By contrast, the thioredoxin, thioredoxin reductase, and thioredoxin peroxidase of the invention are stable in organic solvents, so that enzyme reactions can be carried out in organic solvents or aqueous solutions containing such organic solvents. Thus these proteins or enzymes can act even on the substances which are poorly soluble in aqueous solution, thus broadening the range of applicable reaction materials.

### BRIEF DESCRIPTION OF THE DRAWINGS

FIG. 1(A) is a graph showing the optimal temperature of the thioredoxin reductase derived from Aeropyrum pernix strain K1, and FIG. 1(B) is a graph showing the optimal temperature of the thioredoxin peroxidase derived from Aeropyrum pernix strain K1.

FIG. 2 is a graph showing the heat resistance of thioredoxin, thioredoxin reductase, and thioredoxin peroxidase derived from Aeropyrum pernix strain K1.

FIG. 3(A) is a graph showing the optimal temperature of the thioredoxin derived from Pyrococcus horikoshii OT3 strain, and FIG. 3(B) is a graph showing the optimal temperature of the thioredoxin reductase derived from Pyrococcus horikoshii OT3 strain.

FIG. 4 is a graph showing the optimal temperatures of the thioredoxin and thioredoxin reductase derived from Pyrococcus horikoshii OT3 strain.

### DETAILED DESCRIPTION OF THE INVENTION

The invention is described in detail below.

(1) Proteins or Enzymes of the Invention

Heat Resistance

i) Thioredoxin

The heat resistant thioredoxin of the invention is a protein with highly excellent heat resistance, which retains at least 50% thioredoxin activity after heat treatment at 100° C. for 0.5 hour. It is preferably a protein that retains at least 60% thioredoxin activity after heat treatment at 100° C. for 0.5 hour.

The heat resistant thioredoxin of the invention is more preferably a protein whose thioredoxin activity is substantially unimpaired by heat treatment at 100° C. for 0.5 hour.

In the present invention, “activity is substantially unimpaired” or “substantially no decline in activity” includes, for example, the case of at least 95% residual activity. The maximum temperature at which such a protein exhibits thioredoxin activity is usually about 80° C. Although it depends on the type of buffer in which the reaction is carried out, the enzyme preferably has an optimal temperature of at least 60° C. when determining the initial rate of reaction.

In the present invention, the thioredoxin activity is the value determined by either of methods (1) or (2) described in the Examples.

ii) Thioredoxin Reductase

The heat resistant thioredoxin reductase of the invention is an enzyme with highly excellent heat resistance, which retains at least 50% thioredoxin reductase activity after heat treatment at 100° C. for 0.5 hour. It is preferably an enzyme that retains at least 60% thioredoxin reductase activity after heat treatment at 100° C. for 0.5 hour. The maximum temperature at which activity is manifested is usually about 85° C. Although it depends on the type of buffer in which the reaction is carried out, the enzyme preferably has an optimal temperature of at least 70° C. when determining the initial rate of the enzyme reaction.

The heat resistant thioredoxin reductase of the invention is more preferably an enzyme whose thioredoxin reductase activity is substantially unimpaired by heat treatment at 100° C. for 0.5 hour. The maximum temperature at which such an enzyme exhibits activity is usually about 80° C. Although it depends on the type of buffer in which the enzyme reaction is carried out, the enzyme preferably has an optimal temperature of at least 60° C. when determining the initial rate of the enzyme reaction.

In the present invention, the thioredoxin reductase activity is the value determined by either of methods (1) or (2) described in the Examples.

iii) Thioredoxin Peroxidase

The heat resistant thioredoxin peroxidase of the invention is an enzyme with highly excellent heat resistance, which retains at least 50% thioredoxin peroxidase activity after heat treatment at 100° C. for 0.5 hour. It is preferably an enzyme that retains at least 60% thioredoxin peroxidase activity after heat treatment at 100° C. for 0.5 hour. The maximum temperature at which the heat resistant thioredoxin peroxidase of the invention exhibits activity is usually about 85° C. Although it depends on the type of buffer in which the reaction is carried out, the enzyme preferably has an optimal temperature of at least 70° C. when determining the initial rate of the enzyme reaction.

In the present invention, the thioredoxin peroxidase activity is the value determined by the methods described in the Examples.

Stability at Room Temperature

i) Thioredoxin

The thioredoxin of the invention has excellent stability at room temperature. For example, the thioredoxin of the invention may be a protein that retains at least 90% activity when incubated in 50 mM potassium phosphate buffer (pH 7.0) for 12 hours at 30° C.

ii) Thioredoxin Reductase

The thioredoxin reductase of the invention has excellent stability at room temperature. For example, the thioredoxin reductase of the invention may be an enzyme that retains at least 90% thioredoxin reductase activity after incubation in 50 mM potassium phosphate buffer (pH 7.0) at 30° C. for 12 hours.

iii) Thioredoxin Peroxidase

The thioredoxin peroxidase of the invention has excellent stability at room temperature. For example, the thioredoxin peroxidase of the invention may be an enzyme that retains at least 90% thioredoxin peroxidase activity after incubation in 50 mM potassium phosphate buffer (pH 7.0) at 30° C. for 12 hours.

Organic Solvent Resistance

The thioredoxin, thioredoxin reductase, and thioredoxin peroxidase of the invention are resistant to organic solvents. For example, they may be proteins or enzymes (hereinafter referred to as “proteins”) showing activity in a buffer containing 30 vol % or more of an organic solvent such as ethanol, butanol, tetrahydrofuran, or ethyl acetate. The maximum volumetric percentage of organic solvent in the buffer at which the proteins of the invention can exhibit activity is within the range that the proteins will not precipitate. The present inventors found that this resistance to organic solvents is a characteristic feature of enzymes derived from archaea.

Activity

i) Thioredoxin

The thioredoxin of the invention is a protein which, in its reduced form, is capable of reducing the cysteine residue disulfides of various proteins to dithiols. The thioredoxin of the invention may be a protein which, in its reduced form, is capable of reducing peroxides.

ii) Thioredoxin Reductase

The thioredoxin reductase of the invention is an enzyme capable of converting oxidized thioredoxin to reduced thioredoxin by reducing disulfide to dithiol. The thioredoxin reductase of the invention may be an enzyme capable of reducing oxidized glutathione, etc. Coenzymes such as NADPH, NADH, FADH, and FADH₂ are usually used in such reduction reactions. The coenzyme is preferably used in a proportion of about 100 to 100,000 mols per molecule of thioredoxin reductase.

iii) Thioredoxin Peroxidase

The thioredoxin peroxidase of the invention is an enzyme capable of converting hydrogen peroxide to water in the presence of reduced thioredoxin. The thioredoxin peroxidase of the invention may also be capable of reducing other peroxides. The thioredoxin peroxidase of the invention may also be capable of reducing active oxygen.

Microorganisms Producing the Proteins

Examples of the thioredoxin, thioredoxin reductase, and thioredoxin peroxidase of the invention include proteins produced by microorganisms such as Pyrococcus, Aeropyrum, Sufolobus, Thermoplasma, Thermoproteus, Mastigocladus, Bacillus, Synechococcus, and Thermus.

Of these, proteins produced by hyperthermophilic archaea of genus Aeropyrum, particularly Aeropyrum pernix, are preferred because of their excellent heat resistance. Proteins produced by genus Pyrococcus, particularly Pyrococcus horikoshii, are more preferable because of their further excellent heat resistance.

Thioredoxin and thioredoxin reductase with highly excellent heat resistance that is substantially unimpaired by heat treatment at 100° C. for 0.5 hour are produced, for example, by microorganisms of genus Pyrococcus, particularly Pyrococcus horikoshii.

Amino Acid Sequence

i) Thioredoxin

Examples of the thioredoxin of the invention include polypeptides having the amino acid sequence of (1-1) or (1-2) below:

(1-1) polypeptides comprising the amino acid sequence of SEQ ID NO. 2; and

(1-2) polypeptides comprising the amino acid sequence of SEQ ID NO. 2 with 1 or more amino acids deleted, substituted, or added, and having at least 50% residual thioredoxin activity after heat treatment at 100° C. for 0.5 hour.

In the invention, “the amino acid sequence with 1 or more amino acids deleted, substituted, or added” is preferably one in which no more than 30%, and preferably no more than 10%, of the amino acids in the amino acid sequence have been deleted, substituted, or added.

Examples of “polypeptides comprising the amino acid sequence” in the invention include polypeptides of a length no greater than 3 times that of the amino acid sequence.

Of the polypeptides of (1-1), preferred is a polypeptide consisting of the amino acid sequence of SEQ ID NO. 2. Of the polypeptides of (1-2), preferable are those consisting of the amino acid sequence of SEQ ID NO. 2 with 1 or more amino acids deleted, substituted, or added, and having at least 50% residual thioredoxin activity after heat treatment at 100° C. for 0.5 hour.

Examples of the thioredoxin of the invention also include polypeptides of (2-1) or (2-2) below:

(2-1) polypeptides comprising the amino acid sequence of SEQ ID NO. 8; and

(2-2) polypeptides comprising the amino acid sequence of SEQ ID NO. 8 with 1 or more amino acids deleted, substituted, or added and showing substantially no decline in thioredoxin activity when heat treated at 100° C. for 0.5 hour.

Of the polypeptides of (2-1), preferred is a polypeptide consisting of the amino acid sequence of SEQ ID NO. 8. Of the polypeptides of (2-2), preferred are those consisting of the amino acid sequence of SEQ ID NO. 8 with 1 or more amino acids deleted, substituted, or added and showing substantially no decline in thioredoxin activity when heat treated at 100° C. for 0.5 hour.

In order to obtain polypeptides of (1-2) and (2-2) by modifying polypeptides of (1-1) and (2-1) without causing the loss of biological functions, for example, regions that are not conserved among thioredoxins can be modified. In the unconserved regions, for example, up to 30% of the total number of amino acids can be deleted, substituted, or added.

Specifically, for example, in the case of substitution, amino acids can be substituted with those having similar properties in terms of polarity, charge, solubility, hydrophilicity/hydrophobicity and the like so as to maintain the structure of the protein. For example, amino acids can be substituted with those of the same group shown below. Glycine, alanine, valine, leucine, isoleucine, and proline are classified as nonpolar amino acids; serine, threonine, cysteine, methionine, asparagine, and glutamine are classified as polar amino acids; phenylalanine, tyrosine, and tryptophan are classified as amino acids with aromatic side chains; lysine, arginine, and histidine are classified as basic amino acids; and aspartic acid and glutamic acid are classified as acidic amino acids.

ii) Thioredoxin Reductase

Examples of the thioredoxin reductase of the invention include polypeptides having the amino acid sequence of (3-1) or (3-2) below:

(3-1) polypeptides comprising the amino acid sequence of SEQ ID NO. 4; and

(3-2) polypeptides comprising the amino acid sequence of SEQ ID NO. 4 with 1 or more amino acids deleted, substituted, or added, and having at least 50% residual thioredoxin reductase activity after heat treatment at 100° C. for 0.5 hour.

Of the polypeptides of (3-1), preferred is a polypeptide consisting of the amino acid sequence of SEQ ID NO. 4. Of the polypeptides of (3-2), preferred are those consisting of the amino acid sequence of SEQ ID NO. 4 with 1 or more amino acids deleted, substituted, or added, and having at least 50% residual thioredoxin reductase activity after heat treatment at 100° C. for 0.5 hour.

Examples of the thioredoxin reductase of the invention further include polypeptides having the amino acid sequence of (4-1) or (4-2) below:

(4-1) polypeptides comprising the amino acid sequence of SEQ ID NO. 10; and

(4-2) polypeptides comprising the amino acid sequence of SEQ ID NO. 10 with 1 or more amino acids deleted, substituted, or added, and showing substantially no decline in thioredoxin reductase activity when heat treated at 100° C. for 0.5 hour.

Of the polypeptides of (4-1), preferred is a polypeptide consisting of the amino acid sequence of SEQ ID NO. 10. Of the polypeptides of (4-2), preferred are those consisting of the amino acid sequence of SEQ ID NO. 10 with 1 or more amino acids deleted, substituted, or added, and showing substantially no decline in thioredoxin reductase activity when heat treated at 100° C. for 0.5 hour.

The methods described above can be used to modify the polypeptides of (3-1) and (4-1) without causing the loss of biological functions so as to obtain the polypeptides of (3-2) and (4-2), respectively.

iii) Thioredoxin Peroxidase

Examples of the thioredoxin peroxidase of the invention include polypeptides having the amino acid sequence of (5-1) or (5-2) below:

(5-1) polypeptides comprising the amino acid sequence of SEQ ID NO. 6; and

(5-2) polypeptides comprising the amino acid sequence of SEQ ID NO. 6 with 1 or more amino acids deleted, substituted, or added, and having at least 50% residual thioredoxin peroxidase activity after heat treatment at 100° C. for 0.5 hour.

Of the polypeptides of (5-1), preferred is a polypeptide consisting of the amino acid sequence of SEQ ID NO. 6. Of the polypeptides of (5-2), preferred are those consisting of the amino acid sequence of SEQ ID NO. 6 with 1 or more amino acids deleted, substituted, or added, and having at least 50% residual thioredoxin peroxidase activity after heat treatment at 100° C. for 0.5 hour.

The methods described above can be used to modify the polypeptides of (5-1) without causing the loss of biological functions so as to obtain the polypeptides of (5-2).

Methods for Producing Proteins of the Invention

The thioredoxin, thioredoxin reductase, and thioredoxin peroxidase of the invention can be obtained by culturing microorganisms that produce these proteins and purifying the culture supernatant. The thioredoxin, thioredoxin reductase, and thioredoxin peroxidase can also be obtained by chemical synthesis based on the amino acid sequences of SEQ ID NO. 2 or 8, SEQ ID NO. 4 or 10, and SEQ ID NO. 6, respectively. These proteins can also be obtained by methods of the invention described below.

(2) DNA of the Invention

i) Thioredoxin

Examples of the DNA encoding thioredoxin in the invention include DNAs encoding the polypeptides of (1-1) or (1-2) of the invention as described above. Of these, the DNAs of (6-1) or (6-2) below are preferred:

(6-1) DNAs comprising the base sequence of SEQ ID NO. 1; and

(6-2) DNAs comprising DNAs which hybridize under stringent conditions with DNA consisting of the base sequence of SEQ ID NO. 1 and encode polypeptides having at least 50% residual thioredoxin activity after heat treatment at 100° C. for 0.5 hour.

In the present invention, “DNA which hybridizes under stringent conditions with a designated DNA” preferably has a base sequence encoding a polypeptide whose amino acid sequence is such that no more than 30%, especially no more than 10%, of amino acids of the polypeptide encoded by the designated DNA is deleted, substituted, or added. Examples of “DNA comprising a designated DNA” in the invention include DNA of a length no greater than 3 times that of the designated DNA.

Of the DNAs of (6-1), preferred is DNA consisting of the base sequence of SEQ ID NO. 1. Of the DNAs of (6-2), preferred are those which hybridize under stringent conditions with DNA consisting of the base sequence of SEQ ID No. 1, and encode polypeptides having at least 50% residual thioredoxin activity after heat treatment at 100° C. for 0.5 hour.

Examples of the DNA encoding thioredoxin of the invention also include DNAs encoding the polypeptides of (2-1) or (2-2) of the invention as described above. Of these, DNAs of (7-1) or (7-2) below are preferred:

(7-1) DNAs comprising the base sequence of SEQ ID NO. 7: and

(7-2) DNAs comprising DNAs which hybridize under stringent conditions with DNA consisting of the base sequence of SEQ ID NO. 7 and encode polypeptides showing substantially no decline in thioredoxin activity when heat treated at 100° C. for 0.5 hour.

Of the DNAs of (7-1), preferred is DNA consisting of the base sequence of SEQ ID NO. 7. Of the DNAs of (7-2), preferred are DNAs which hybridize under stringent conditions with DNA consisting of the base sequence in SEQ ID NO. 7 and encode polypeptides showing substantially no decline in thioredoxin activity when heat treated at 100° C. for 0.5 hour.

In order to obtain polypeptides of (6-2) and (7-2) by modifying polypeptides of (6-1) and (6-1) without causing the loss of biological functions, any region that is not conserved among thioredoxins can be modified. Up to 30% of the total number of nucleotides can be deleted, substituted, or added, provided that such modification is made in the unconserved regions.

ii) Thioredoxin Reductase

Examples of the DNA encoding thioredoxin reductase in the invention include DNAs encoding the polypeptides of (3-1) or (3-2) of the invention as described above. Of these, the DNAs of (8-1) or (8-2) below are preferred:

(8-1) DNAs comprising the base sequence of SEQ ID NO. 3; and

(8-2) DNAs comprising DNAs which hybridize under stringent conditions with DNA consisting of the base sequence of SEQ ID NO. 3 and encode polypeptides having at least 50% residual thioredoxin reductase activity after heat treatment at 100° C. for 0.5 hour.

Of the DNAs of (8-1), preferred is DNA consisting of the base sequence of SEQ ID NO. 3. Of the DNAs of (8-2), preferred are DNAs which hybridize under stringent conditions with DNA consisting of the base sequence in SEQ ID NO. 3 and encode polypeptides having at least 50% residual thioredoxin reductase activity after heat treatment at 100° C. for 0.5 hour.

Examples of the DNA encoding thioredoxin reductase in the invention also include DNAs encoding the polypeptides of (4-1) or (4-2) of the invention as described above. Of these, the DNAs of (9-1) or (9-2) below are preferred:

(9-1) DNAs comprising the base sequence of SEQ ID NO. 9; and

(9-2) DNAs comprising DNAs which hybridize under stringent conditions with DNA consisting of the base sequence of SEQ ID NO. 9 and encode polypeptides showing substantially no decline in thioredoxin reductase activity when heat treated at 100° C. for 0.5 hour.

Of the DNAs of (9-1), preferred is DNA consisting of the base sequence of SEQ ID NO. 9. Of the DNAs of (9-2), preferred are DNAs that hybridize under stringent conditions with DNA consisting of the base sequence of SEQ ID NO. 9, and encode polypeptides showing substantially no decline in thioredoxin reductase activity when heat treated at 100° C. for 0.5 hour.

The methods described above can be used to modify the DNAs of (8-1) and (9-1) without causing the loss of biological functions so as to obtain the polypeptides of (8-2) and (9-2), respectively.

iii) Thioredoxin Peroxidase

Examples of the DNA encoding thioredoxin peroxidase in the invention include DNAs encoding polypeptides of (5-1) or (5-2) of the invention as described above. Of these, DNAs of (10-1) or (10-2) below are preferred:

(10-1) DNAs comprising the base sequence of SEQ ID NO. 5; and

(10-2) DNAs comprising DNAs which hybridize under stringent conditions with DNA consisting of the base sequence of SEQ ID NO. 5 and encode polypeptides having at least 50% residual thioredoxin peroxidase activity after heat treatment at 100° C. for 0.5 hour.

Of the DNAs of (10-1), preferred is DNA consisting of the base sequence of SEQ ID NO. 5. Of the DNAs of (10-2), preferred are DNAs which hybridize under stringent conditions with DNA consisting of the base sequence of SEQ ID NO. 5, and encode polypeptides having at least 50% residual thioredoxin peroxidase activity after heat treatment at 100° C. for 0.5 hour.

The methods described above can be used to modify the DNA of (10-1) without causing the loss of biological functions so as to obtain the DNA of (10-2).

Stringent Conditions

In this specification, examples of “stringent conditions” include the conditions of 68° C. in an ordinary hybridization solution, and the conditions of 42° C. in a hybridization solution containing 50% formamide. Specific examples include the conditions used for Southern hybridization as described in “Molecular Cloning: A Laboratory Manual”, 2nd Edition, Volume 2.

Method for Producing DNA of the Invention

DNA encoding the proteins of the invention can be isolated by hybridization with the use of a probe from a chromosomal DNA library of thermophilic archaea such as genuses Pyrococcus, Aeropyrum, Sufolobus, Thermoplasma, Thermoproteus, Mastigocladus, Bacillus, Synechococcus, and Thermus. The DNA of the invention can be amplified by PCR using chromosomal DNA libraries of these microorganisms as templates. Probes and primers for the DNA encoding thioredoxin of (6-1) or (7-2), DNA encoding thioredoxin reductase of (8-1) or (9-1), and DNA encoding the thioredoxin peroxidase of (10-1) can be designed based on the DNA sequences of SEQ ID NO. 1 or 7, SEQ ID NO. 3 or 9, and SEQ ID NO. 5, respectively. The probes and primers can also be obtained by chemical synthesis.

The DNA variants of (6-2), (7-2), (8-2), (9-2), and (10-2) can be prepared by known methods such as chemical synthesis, genetic engineering, and mutagenesis. Examples of genetic engineering include the alternation of available thioredoxin, thioredoxin reductase or thioredoxin peroxidase by known methods such as the introduction of DNA deletions using exonucleases, the introduction of linkers, site-directed mutagenesis, and the modification of base sequences by PCR using variant primers.

(3) Vectors of the Invention

The vectors of the invention are recombinant vectors comprising the DNA of the invention described above. A wide range of known vectors can be used to be integrated with the DNA of the invention. Vectors for bacteria, yeasts, and animal cells can be used. For the sake of efficient enzyme production, vectors for bacteria are usually used. Examples of well known vectors include E. coli vectors such as pBR322, pUC19, and pKK233-2, genus Bacillus vectors such as pUB110, pC194, pE194, pTHT15, and pBD16, vectors for yeasts such as Yip5, Yrp17, and Yep24, and vectors for animal cells such as pUC18, pUC19, and M13mp18.

(4) Transformants of the Invention

Transformants of the invention are transformants comprising the recombinant vectors of the invention as described above. Bacterial cells, yeasts, animal cells, and the like can be used as hosts, which can be selected depending on the desired vector. Bacillus subtilis, Bacillus brevis, yeasts, fungi and the like are preferred as the host to enable mass production of the target proteins.

Transformation can be brought about by a known method such as the calcium phosphate method, protoplast method, electroporation, spheroplast method, lithium acetate method, lipofection, and microinjection. A method suitable to the type of host can be selected from such known methods.

(5) Method for Producing Proteins or Enzymes of the Invention

The methods for producing the thioredoxin, thioredoxin reductase, and thioredoxin peroxidase in the invention are methods for culturing transformants of the invention and then collecting proteins from the resulting transformants. Intracellularly or intraperiprasmically produced proteins of the invention can be collected by rupturing the cells by a known method such as ultrasonic treatment or surfactant treatment. Proteins of the invention secreted in a culture broth can be collected by isolating the culture broth and optionally concentrating the same.

The collected proteins can be purified by a combination of known protein purification methods, such as centrifugation, salting out, precipitation by solvent, dialysis, ultrafiltration, gel filtration, ion exchange chromatography, affinity chromatography, and reversed phase HPLC.

When the heat resistant proteins of the invention are purified, the purification process preferably comprises an incubation step in which a solution of the proteins to be purified is incubated, usually for about 10 to 120 minutes, and particularly about 10 to 30 minutes, at a temperature such that incubating the protein for about 10 to 30 minutes (particularly about 20 minutes) normally results in at least 60% and particularly at least 80% residual activity, and that is usually at least 10° C., particularly at least 15° C., higher than the critical growth temperature of the host producing the protein. This allows protein impurities produced by the hosts to be denatured or inactivated, with virtually no loss of target protein activity. After the heat treatment step, the protein solution can be centrifuged, for example, at about 15,000 rpm for about 20 minutes, although not limited thereto, to allow the denatured protein impurities to be precipitated. This heat treatment step may be implemented at any stage of the purification process.

Such a heat treatment step can be implemented not only for the purification of the thioredoxin, thioredoxin reductase, and thioredoxin peroxidase of the invention, but for the purification of any heat resistant protein, thereby dramatically improving the purity of heat resistant proteins.

### EXAMPLES

The present invention is illustrated in the following examples and test examples, but the present invention is not limited to these examples.

Assay of Activity

The following methods were employed to detect the target proteins or enzymes in the purification process and to assay the activity of the target proteins or enzymes in order to study the optimal temperature and stability.

i) Assay of Thioredoxin Activity (1)

The activity of thioredoxin derived from Aeropyrum pernix was assayed in accordance with the method of Holmgren et al (Method in Enzymology (1993)) for assaying activity of reducing disulfide bonds between insulin subunits by reduced thioredoxin.

Specifically, a thioredoxin sample was pretreated for 15 minutes at 37° C. in 100 mM Tris-HCl buffer (pH 7.5) containing 0.4 mM DTT and 0.4 mg/ml bovine serum albumin to produce reduced thioredoxin. Then 200 ng of thioredoxin was added to 100 mM Tris-HCl buffer (pH 7.5) containing 1 mg/ml bovine spleen insulin (product of Sigma) as substrate, and the increase in absorbance at 650 nm resulting from the reduction and degradation of the insulin was determined at room temperature for 20 minutes.

The activity was assayed in 70° C. buffer during the purification process.

ii) Assay of Thioredoxin Activity (2)

The activity of thioredoxin derived from Pyrococcus horikoshii was assayed in the same manner as in the thioredoxin activity assay method (1) except that thioredoxin was added in an amount of 25,000 ng. The activity was assayed in 60° C. buffer during the purification process.

iii) Assay of Thioredoxin Reductase Activity (1)

The activity of thioredoxin reductase derived from Aeropyrum pernix was assayed in the following manner. 2 μg of thioredoxin reductase was added to 50 mM potassium phosphate buffer (pH 7.0) containing 1 mM dithiobis(2-nitrobenzoic acid) (DTNB) as substrate, 0.2 mM NADPH and 1 mM EDTA, and the rate at which the absorbance at 340 nm decreases (indicator of NADPH concentration) was determined for 5 minutes so as to assay the thioredoxin reductase activity.

The activity was assayed in 70° C. buffer during the purification process.

iv) Assay of Thioredoxin Reductase Activity (2)

The activity of thioredoxin reductase derived from Pyrococcus horikoshii was assayed in the following manner. 2 μg of thioredoxin reductase was added to 50 mM potassium phosphate buffer (pH 7.0) containing 0.5 mM dithiobis(2-nitrobenzoic acid) (DTNB) as substrate, 0.2 mM NADPH and 1 mM EDTA, and the rate at which the absorbance at 412 nm increases (indicator of TNB (DTNB decomposition product) concentration) was assayed for 1 minute so as to assay the thioredoxin reductase activity.

The activity was assayed in 60° C. buffer during the purification process.

v) Assay of Thioredoxin Peroxidase Activity (2)

2 μg of thioredoxin peroxidase was added to 50 mM potassium phosphate buffer (pH 7.0) containing 1 mM hydrogen peroxide as substrate, 0.2 mM NADPH, 0.1 μM purified thioredoxin reductase and 5 μM purified thioredoxin, and the rate at which the absorbance at 340 nm decreases (indicator of NADPH concentration) was determined for 5 minutes so as to assay the thioredoxin peroxidase activity.

The activity was assayed in 70° C. buffer during the purification process.

Protein Derived from Hyperthermophilic Archaea Aeropyrum pernix Strain K1

### Example 1-1

(Culture of Aeropyrum pernix strain K1)

Medium was prepared by dissolving 37.4 g of Bacto Marine medium (Difco) and 1.0 g of Na₂S₂O₃.5H₂O in 1 liter of water, and then adjusting the pH to 7.0 to 7.2. A hyperthermophilic archaeon Aeropyrum pernix strain K1 (registered as JCM9820 at The Institute of Physical and Chemical Research) was inoculated into the medium and cultured with shaking at 90° C. for 3 days. The culture broth was centrifuged at 5,000 rpm for 10 minutes to harvest the microorganisms.

### Example 1-2

(Preparation of Chromosomal DNA)

The microbial cells were washed twice with 10 mM Tris (pH 7.5)-1 mM EDTA solution, and then sealed in InCert Agarose blocks (product of FMC). The blocks were treated with 1% N-lauroylsarcosine-1 mg/ml protease K solution, allowing the chromosomal DNA to be isolated in the agarose blocks. The conditions under which the chromosomal DNA was isolated using the InCert Agarose blocks were in accordance with the manual accompanying the agarose blocks.

### Example 1-3

(Construction of Expression Plasmids)

i) Thioredoxin

DNA comprising the base sequence of SEQ ID NO. 1 was amplified by PCR in the following manner. The PCR conditions were in accordance with the manual accompanying the PCR kit. An oligonucleotlde primer beginning from the first base (that is, beginning from the start codon) in the DNA sequence of SEQ ID NO. 1 was synthesized as a primer for the 5′ end. A primer corresponding to the region downstream from the 3′ end of the base sequence of SEQ ID NO. 1 in the chromosomal DNA of Aeropyrum pernix K1, which was a primer producing the restriction enzyme BamHI site in the amplified DNA, was synthesized as primer for the 3′ end. After the PCR reaction, the amplified DNA was treated with the BamHI restriction enzyme at 37° C. for 3 hours and thus completely degraded digested. The thioredoxin gene was then purified using a purification column kit.

To construct a vector containing the thioredoxin gene insert, the pET-8c vector (product of Novagen) was then cleaved with NcoI restriction enzyme and purified, and the ends were blunted using T4 DNA polymerase. The purified plasmid was cleaved and purified with BamHI restriction enzyme. The pET-8c plasmid cleaved with BamHI and the aforementioned thioredoxin gene cleaved with BamNHI were then ligated by 16 hours of reaction with T4 ligase at 16° C. The ligated DNA was used to transform competent cells of the E. coli XL2-BlueMRF′ strain (product of Stratagene). Transformants were selected on the basis of the formation of colonies on LB agar plates containing 0.05 mg/mL ampicillin. Plasmids containing the thioredoxin gene were extracted from the transformants by the alkali method.

ii) Thioredoxin Reductase

An oligonucleotide beginning from the first base in the DNA sequence of SEQ ID NO. 3 was synthesized as the PCR primer for the 5′ end. A primer corresponding to the downstream side from the 3′ end of the base sequence of SEQ ID NO. 3 in the chromosomal DNA of Aeropyrum pernix K1, which was a primer producing the BamHI site in the amplified DNA, was synthesized as the PCR primer corresponding to the 3′ end. An E. coli XL2-BlueMRF′ strain with the pET-8c plasmid comprising the thioredoxin reductase derived from Aeropyrum pernix K1 was obtained in the same manner as for the thioredoxin above. Plasmids containing the thioredoxin reductase gene were extracted from the transformants by the alkali method.

iii) Thioredoxin Peroxidase

An oligonucleotide beginning from the first base in the DNA sequence of SEQ ID NO. 5 was synthesized as the PCR primer for the 5′ end. A primer corresponding to the downstream side from the 3′ end of the nucleotide sequence of SEQ ID NO. 5 in the chromosomal DNA of Aeropyrum pernix K1, which was a primer producing the BamHI site in the amplified DNA, was synthesized as the PCR primer corresponding to the 3′ end. An E. coli XL2-BlueMRF′ strain with the pET-8c plasmid comprising the thioredoxin peroxidase derived from Aeropyrum pernix K1 was obtained in the same manner as for the thioredoxin above. Plasmids containing the thioredoxin peroxidase gene were extracted from the transformants by the alkali method.

### Example 1-4

(Preparation of Transformants)

To 1.5 ml tubes were added 0.04 ml (20,000,000 cfu/μg) competent cells of the E. coli Rosetta (DE3) strain (product of Novagen) and 0.003 ml DNA solution (8.4 ng plasmid DNA) of plasmids containing the thioredoxin gene, thioredoxin reductase gene or thioredoxin peroxidase gene prepared in Example 1-4. The tubes were allowed to stand in ice for 30 minutes, and heat shock was then given for 30 seconds at 42° C. 0.25 ml SOC medium was then added to the tubes and cultured with shaking at 37° C. for 1 hour. LB agar plates containing ampicillin and chloramphenicol were then smeared with the microbial cell culture and cultured at 37° C. overnight, giving transformants.

### Example 1-5

(Proteins or Enzymes Purification)

i) Thioredoxin

Transformants having plasmids containing the thioredoxin gene were inoculated into NZCYM medium containing ampicillin and chloramphenicol and cultured at 37° C. until the absorbance at 600 nm reached 0.5. IPTG (isopropyl-â-D-thiogalactopyranoside) was added to enhance the amount of plasmid expression, and the transformants were cultured for another 4 hours. The culture broth was centrifuged at 8,000 rpm for 10 minutes to harvest the microbial cells.

50 mM Tris-HCl (pH 8.0) containing 1 mM DTT and 1 mM EDTA was added to 4 g of the harvested microbial cells, and the cells were ultrasonically ruptured for 5 minutes at an output power of 90 W. The ruptured cells were centrifuged at 15,000 rpm for 30 minutes, and the supernatant was collected.

To remove protein contaminants by precipitation, the supernatant was heated at 85° C. for 30 minutes and then centrifuged at 15,000 rpm for 30 minutes and the supernatant was collected. The supernatant was dialyzed against 50 mM (Tris-HCl) buffer (pH 8.0) containing 1 mM EDTA, followed by ion exchange chromatography on a Hitrap Q (product of Pharmacia) column of anion exchange resin equilibrated with the same buffer. The active fractions were dialyzed against 50 mM sodium phosphate buffer (pH 7.0) containing 150 mM NaCl and thus equilibrated with the same buffer. The resulting protein solution was subjected to gel filtration chromatography on a column of Superdex 200 (product of Pharmacia). The resulting active fractions contained a homogenous preparation giving a single band by SDS-PAGE.

Gel filtration chromatography revealed that the enzyme had a molecular weight of about 37 kDa.

ii) Thioredoxin Reductase

Transformants with plasmids containing the thioredoxin reductase gene were cultured to harvest cells in the same manner as for the thioredoxin gene.

50 mM Tris-HCl (pH 8.5) containing 1 mM DTT and 1 mM EDTA was added to 4 g of the harvested microbial cells, and the cells were ultrasonically ruptured for 5 minutes at an output power of 90 W. The ruptured cells were centrifuged at 15,000 rpm for 30 minutes, and the supernatant was collected.

To remove protein contaminants by precipitation, the supernatant was heated at 85° C. for 30 minutes and then centrifuged at 15,000 rpm for 20 minutes, and the supernatant was collected. The supernatant was dialyzed against 50 mM (Tris-HCl) buffer (pH 8.0) containing 1 mM EDTA, followed by ion exchange chromatography on a Hitrap Q (product of Pharmacia) column of anion exchange resin equilibrated with the same buffer. The active fractions were dialyzed against 50 mM sodium phosphate buffer (pH 7.0) containing 150 mM NaCl and thereby equilibrated with the same buffer. The resulting protein solution was subjected to gel filtration chromatography on a Superdex 200 (product of Pharmacia) column. The active fractions contained a homogenous preparation giving a single band by SDS-PAGE.

Gel filtration chromatography revealed that the enzyme had a molecular weight of about 37 kDa.

iii) Thioredoxin Peroxidase

Transformants with plasmids containing the thioredoxin peroxidase gene were cultured, harvested, sonicated, and centrifuged to obtain a supernatant, which was then heat treated and centrifuged to obtain a supernatant, in the same manner as for the thioredoxin gene.

The resulting supernatant was dialyzed against 50 mM (Tris-HCl) buffer (pH 8.0) containing 1 mM EDTA, followed by ion exchange chromatography on a Hitrap Q (product of Phanmacia) column of anion exchange resin equilibrated with the same buffer. The active fractions were further dialyzed against 50 mM sodium phosphate buffer (pH 7.0) containing 150 mM NaCl and thereby equilibrated with the same buffer. The resulting protein solution was applied on a Sephacryl S-100 (product of Pharmacia) column for gel filtration chromatography. The active fractions contained a homogenous preparation giving a single band by SDS-PAGE.

Gel filtration chromatography revealed that the enzyme had a molecular weight of about 29 kDa.

### Example 1-5

(Sequencing of Base Sequence and Amino Acid Sequence)

The base sequences of the thioredoxin, thioredoxin reductase and thioredoxin peroxidase obtained in Example 1-4 are shown in SEQ ID NOS. 1, 3, and 5. The amino acid sequences are shown in SEQ ID NOS. 2, 4, and 6.

A homology search by computer revealed 31% homology between the base sequence of SEQ ID NO. 1 of the thioredoxin gene derived from the Aeropyrum pernix K1 strain and the base sequence of the thioredoxin gene of the Salmonella typhimurium LT2 strain. 48% homology was found between the base sequence of SEQ ID NO. 3 of the thioredoxin reductase gene derived from the Aeropyrum pernix K1 strain and the base sequence of the thioredoxin reductase gene of Sulfolobus solfataricus. 62% homology was found between the base sequence of SEQ ID NO. 5 of the thioredoxin peroxidase gene derived from the Aeropyrum pernix K1 strain and the base sequence of the thioredoxin peroxidase gene of Sulfolobus tokodaii.

### Example 1-6

(Optimal Temperature)

The optimal temperatures for Aeropyrum pernix K1-derived thioredoxin, thioredoxin reductase and thioredoxin peroxidase obtained in Example 1-4 were evaluated.

The temperature of the buffer in which the enzyme reactions were carried out in the aforementioned assay (1) of thioredoxin reductase activity and the assay (1) of thioredoxin peroxidase activity were varied over the range of 20 to 90° C. to assay the activity of the thioredoxin reductase and thioredoxin peroxidase.

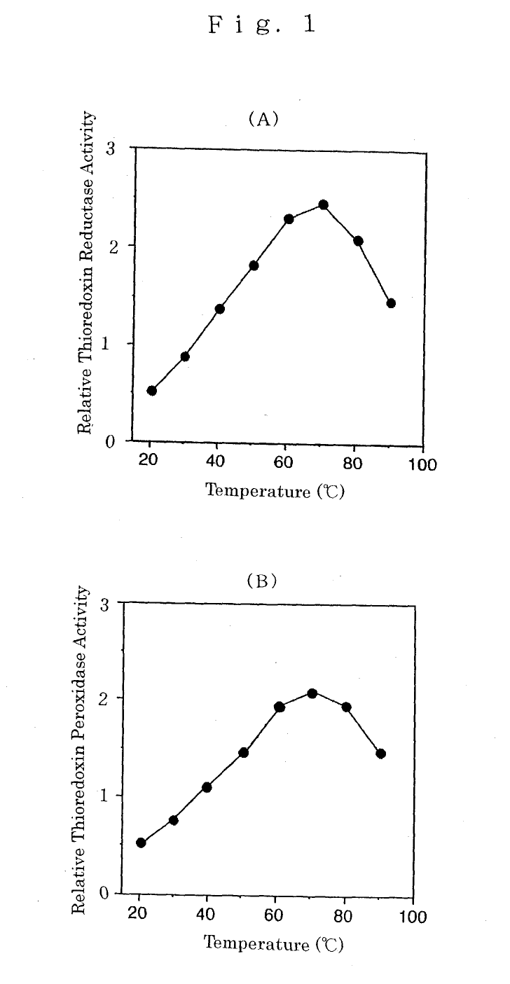

As shown in FIG. 1, the optimal temperature for thioredoxin reductase was 70° C. (FIG. 1(A)), and the optimal temperature for thioredoxin peroxidase was 70° C. (FIG. 1(B)).

### Example 1-8

(Heat Resistance)

Samples of enzyme solution were prepared by adding thioredoxin obtained in Example 1-4 to a concentration of 0.1 mg/mL in 50 mM sodium phosphate buffer (pH 7.0) containing 2 mM EDTA. The samples were incubated at 100° C. to assay the residual activity over time. Thioredoxin reductase and thioredoxin peroxidase obtained in Example 1-4 were similarly incubated to assay the activity over time.

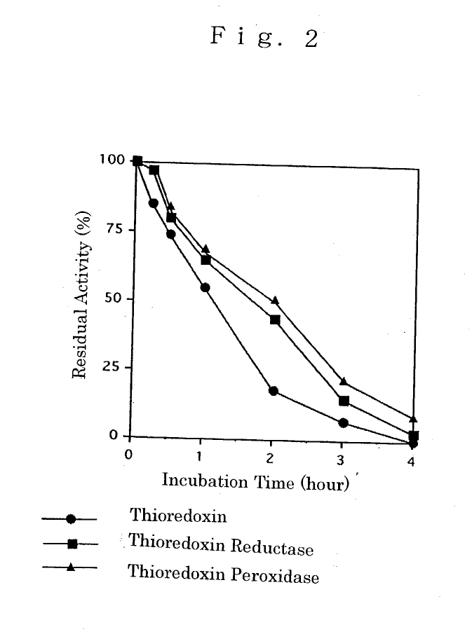

As shown in FIG. 2, incubation of thioredoxin at 100° C. resulted in about 55% residual activity after 1 hour, incubation of thioredoxin reductase at 100° C. resulted in about 65% residual activity after 1 hour, and incubation of thioredoxin peroxidase at 100° C. resulted in about 70% activity after 1 hour.

Protein Derived From Hyperthermophilic Arachaea Pyrococcus horikoshii OT3 strain

### Example 2-1

(Culture of Pyrococcus horikoshii OT3 Strain)

13.5 g of sodium chloride, 4 g of Na₂SO₄, 0.7 g of KCl, 0.2 g of NaHCO₃, 0.1 g of KBr, 30 mg of H₃BO₃, 10 g of MgCl₂.6H₂O, 1.5 g of CaCl₂, 25 mg of SrCl₂, 1.0 mL of resazurin solution (0.2 g/l), 1.0 g of yeast extract and 5 g of bactopeptone were dissolved in 1 liter of water, the pH of the solution was adjusted to 6.8, and the solution was sterilized under pressure. Sulfur which had been sterilized in dry oven was then added to a concentration of 0.2 wt %, the medium was saturated with argon to render it anaerobic and inoculated with Pyrococcus horikoshii OT3 (registered as JCM9974 at the Institute of Physical and Chemical Research). To determine whether or not the medium was anaerobic, Na₂S solution was added to check that the resazurin solution was not colored pink by oxygen in the culture broth. The culture broth was cultured at 95° C. for 2 to 4 days, and then centrifuged at 5000 rpm for 10 minutes to harvest the cells.

### Example 2-2

(Preparation of Chromosomal DNA)

Chromosomal DNA of the Pyrococcus horikoshii OT3 strain was prepared in the following manner. The harvested cells were washed twice with 10 mM Tris (pH 7.5)-1 mM EDTA solution and then sealed in InCert Agarose (product by FMC) blocks. The blocks were treated with 1% N-lauroyl sarcosine-1 mg/ml protease K solution, allowing the chromosomal DNA to be isolated in the agarose blocks.

### Example 2-3

(Construction of Thioredoxin Gene Expression Plasmids)

DNA containing the base sequence of SEQ ID NO. 7 was amplified by PCR using as template the chromosomal DNA of the Pyrococcus horikoshii OT3 strain obtained in Example 2-2. The conditions of the PCR were in accordance with the manual accompanying the PCR kit. DNA primer GGAATTCCATATGGGACTAATAAGTGAGGAGGA (SEQ ID NO. 11) having a restriction enzyme (Ndel) site was synthesized as a primer corresponding to the 5′ end side of the structural gene region. To construct a restriction enzyme (BamHI) site, the DNA primer CGGGATCCTAGCTTAGGGCTGAAAGTAGG (SEQ ID NO. 12) was synthesized as a primer corresponding to the 3′ end side of the structural gene region. After the PCR reaction, the amplified DNA was completely degraded with the restriction enzymes (Ndel and BamHI) (overnight at 37° C.). The thioredoxin gene was then purified using a purification column kit.

The pET-11a vector (product of Novagen) was completely degraded with the restriction enzymes Ndel and BamHI and then purified using a purification column kit. The resulting DNA fragments were ligated to the above thioredoxin structural gene by reaction at 16° C. for 3 hours using T4 DNA ligase. Some of the ligated DNA was introduced into E. coli-XL2-Blue MRF′ competent cells. Transformants were selected on the basis of the formation of colonies on LB agar plates containing ampicillin. Thioredoxin expression plasmids were extracted from the resulting colonies by the alkali method and purified.

### Example 2-4

(Preparation of Transformants Having Thioredoxin Gene)

Competent cells of the E. coli Rosetta (DE3) strain (product of Novagen) were unfreezed, and 0.04 ml of the cells was transferred to a 1.5 ml tube. 0.003 ml of the thioredoxin expression plasmid solution obtained in Example 2-3 was added to the tube, the tubes were allowed to stand in ice for 30 minutes, and heat shock was then given at 42° C. for 30 seconds. 0.25 ml of SOC medium was added to the tubes, followed by culturing with shaking at 37° C. for 1 hour. LB agar plates containing ampicillin were then smeared with the microbial cell culture and cultured overnight at 37° C., giving transformant colonies.

The transformants were cultured in NZCYM medium containing ampicillin until the absorbance at 600 nm reached 0.6, IPTG (isopropyl-â-D-thiogalactopyranoside) was then added, and the transformants were cultured for another 4 hours. The culture broth was centrifuged at 7000 rpm for 5 minutes to harvest the microbial cells.

### Example 2-5

(Purification of Heat Resistant Thioredoxin)

50 mM Tris-HCl (pH 8.0) buffer containing 1 mM DTT and 1 mM EDTA was added to the microbial cells harvested in Example 2-4, and the cells were ultrasonically ruptured. The solution of ruptured cells was centrifuged (at 15,000 rpm for 30 minutes), the supernatant was then collected, the resulting supernatant was heated at 85° C. for 30 minutes and then centrifuged at 15,000 rpm for 30 minutes, and the supernatant was subjected to anion exchange chromatography on Hitrap Q (product of Pharmacia), hydrophobic interaction chromatography on Hiload phenyl, and gel filtration chromatography on Sephacryl S-100 (product of Pharmacia), in that order, resulting in a preparation giving an uniform band by SDS-PAGE.

The molecular weight, as determined by gel filtration chromatography of the resulting preparation, was about 27 kDa (National Institute of Technology and Evaluation; Registration No. PH0178).

### Example 2-6

(Preparation of Heat Resistant Thioredoxin Reductase)

A thioredoxin reductase expression plasmid was produced in the same manner as in Example 2-3 except that PCR was carried out using a chromosomal DNA of the Pyrococcus horikoshii OT3 strain obtained in Example 2-2 as template, GGAATTCCATATGGAGGTGAAGGAAATGTTCA (SEQ ID NO. 13) as a primer corresponding to the 5′ end side of the structural gene, and CGGGATCCTCACTCAATAGTCTTTCCATTCC (SEQ ID NO. 14) as a primer corresponding to the 3′ end side of the structural gene.

This thioredoxin reductase expression plasmid was used to produce a recombinant thioredoxin with the E. coli Rosetta (DE) strain in the same manner as in Example 2-4.

50 mM Tris-HCl (pH 8.5) buffer containing 1 mM DTT and 1 mM EDTA was added to the harvested microbial cells, and the cells were ultrasonically ruptured. The resulting liquid was centrifuged (30 minutes at 15,000 rpm), and the resulting supernatant was then heated at 85° C. for 30 minutes and then centrifuged at 15,000 rpm for 30 minutes. The supernatant was treated by anion exchange chromatography on Hitrap Q (product of Pharmacia), hydrophobic interaction chromatography on Hiload phenyl, and gel filtration chromatography on Superdex 200 (product of Pharmacia), in that order, resulting in a preparation giving an uniform band by SDS-PAGE.

The molecular weight, as determined by SDS-PAGE of the resulting preparation, was about 37 kDa (National Institute of Technology and Evaluation; Registration No. PH1426).

### Example 2 -7

(Sequencing of Base Sequence and Amino Acid Sequence)

The base sequence of the heat resistant thioredoxin derived from the Pyrococcus horikoshii OT3 strain obtained in Example 2-5 is shown in SEQ ID NO. 7, and the amino acid sequence is shown in SEQ ID NO. 8. The base sequence of the heat resistant thioredoxin reductase derived from the same strain obtained in Example 2-6 is shown in SEQ ID NO. 9, and the amino acid sequence is shown in SEQ ID NO. 10.

### Example 2-8

(Optimal Temperature)

The temperature of the buffer in which the enzyme reactions were carried out in the aforementioned assay (2) of thioredoxin activity and the assay (2) of thioredoxin reductase activity were varied over the range of 25 to 65° C. to assay the activity of heat resistant thioredoxin derived from the Pyrococcus horikoshii OT3 strain obtained in Example 2-5 and of thioredoxin reductase derived from the same strain obtained in Example 2-6.

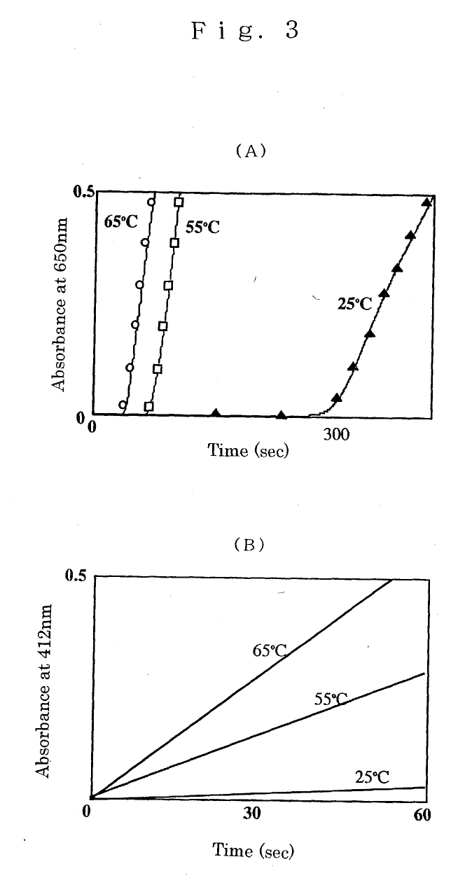

FIG. 3(A) shows the results obtained when the change in absorbance at 650 nm was plotted against the reaction time in the assay of thioredoxin activity. FIG. 3(B) shows the results obtained when the change in absorbance at 412 nm was plotted against the reaction time in the assay of thioredoxin reductase activity. FIGS. 3(A) and 3(B) show that the optimal temperature for thioredoxin was at least 65° C., and that the optimal temperature for thioredoxin reductase was at least 65° C.

### Example 2-2

(Heat Resistance)

A sample of enzyme solution was prepared by adding thioredoxin obtained in Example 2-5 to a concentration of 25 mg/ml in 50 mM sodium phosphate buffer (pH 7.0), and the sample was incubated at 100° C. to assay the residual activity over time. The thioredoxin reductase obtained in Example 2-6 was similarly incubated to assay the activity over time.

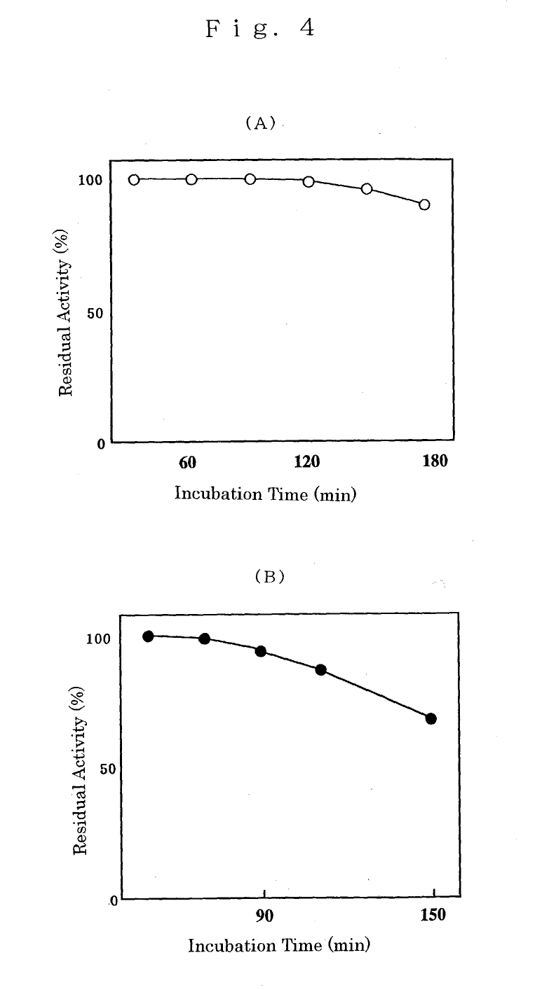

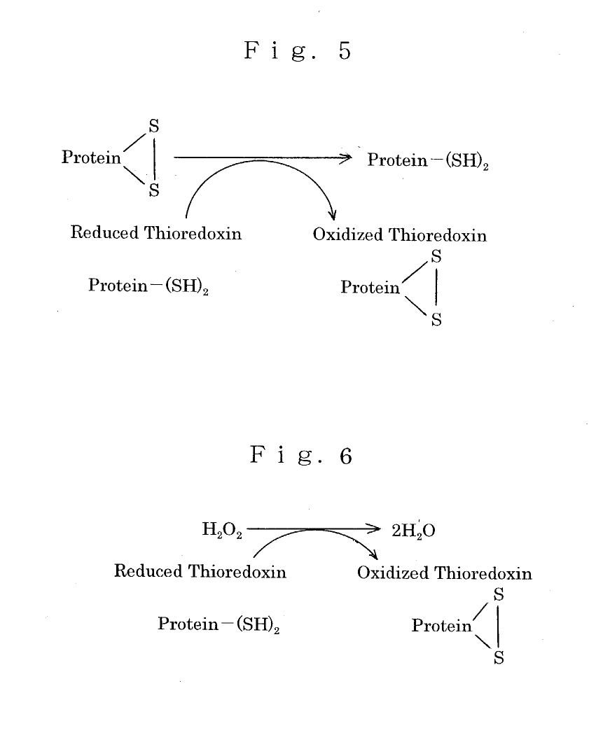

FIGS. 4(A) and 4(B) show the results. FIG. 4(A) shows that the thioredoxin had about 100% residual activity after incubation at 100° C. for 0.5 hour. FIG. 4(B) shows that the thioredoxin reductase had about 99% residual activity after incubation at 100° C. for 0.5 hour.

### INDUSTRIAL APPLICABILITY

The thioredoxin, thioredoxin reductase and thioredoxin peroxidase of the invention have highly excellent heat resistance and thus can be sterilized in heating. These proteins are thus suitable for use as additives in drugs, food products, animal feed, cosmetics, and the like. They are also in themselves suitable for use as the active ingredients of pharmaceuticals, cosmetics, and the like. They are also suitable as reaction reagents for reactions at high temperatures.

What is claimed is:

1. A heat resistant thioredoxin having at least 50% residual activity after heat treatment at 100° C. for 0.5 hour.

2. A heat resistant thioredoxin according to claim 1. derived from hyperthermophilic archaea Aeropyrum pernix.

3. A polypeptide of (1-1) or (1-2) below:
(1-1) a polypeptide comprising the amino acid sequence of SEQ ID NO. 2; and
(1-2) a polypeptide comprising the amino acid sequence of SEQ ID NO. 2 with 1 or more amino acids deleted, substituted, or added, the polypeptide having at least 50% residual thioredoxin activity after heat treatment at 100° C. for 0.5 hour.

4. DNA of (6-1), (6-2) or (6-3) below:
(6-1) DNA comprising the base sequence of SEQ ID NO. 1;
(6-2) DNA comprising DNA which hybridizes under stringent conditions with DNA consisting of the base sequence of SEQ ID NO. 1 and encodes a polypeptide having at least 50% residual thioredoxin activity after heat treatment at 100° C. for 0.5 hour; and
(6-3) DNA encoding the polypeptide according to claim 3

5. A vector comprising the DNA according to claim 4.

6. A transformant comprising the vector according to claim 5.

7. A method for producing a heat resistant thioredoxin, comprising the steps of culturing the transformant of claim 6, and collecting the heat resistant thioredoxin from the transformant.

8. A heat resistant thioredoxin according to claim 1 whose activity is substantially unimpaired by heat treatment at 100° C. for 0.5 hour.

9. A heat resistant thioredoxin according to claim 8, derived from hyperthermophilic archaea Pyrococcus horikoshii.

10. A polypeptide of (2-1) or (2-2) below:
(2-1) a polypeptide comprising the amino acid sequence of SEQ ID NO. 8; and
(2-2) a polypeptide comprising the amino acid sequence of SEQ ID NO. 8 with 1 or more amino acids deleted, substituted, or added, the polypeptide having thioredoxin activity which is substantially unimpaired by heat treatment at 100° C. for 0.5 hour.

11. DNA of (7-1), (7-2) or (7-3) below:
(7-1) DNA comprising the base sequence of SEQ ID NO. 7;
(7-2) DNA comprising DNA which hybridizes under stringent conditions with DNA consisting of the base sequence of SEQ ID NO. 7 and encodes a polypeptide whose thioredoxin activity is substantially unimpaired by heat treatment at 100° C. for 0.5 hour.
(7-3) DNA encoding the polypeptide according to claim 10.

12. A vector comprising the DNA of claim 11.

13. A transformant comprising the vector of claim 12.

14. A method for producing a heat resistant thioredoxin, comprising the steps of culturing the transformant of claim 13 and collecting the heat resistant thioredoxin from the transformant.

15. A heat resistant thioredoxin reductase having at least 50% residual activity after heat treatment at 100 °C. for 0.5 hour.

16. A heat resistant thioredoxin reductase according to claim 15, derived from hyperthermophilic archaea Aeropyrum pernix.

17. A polypeptide of (3-1) or (3-2) below:
(3-1) a polypeptide comprising the amino acid sequence of SEQ ID NO. 4; and
(3-2) a polypeptide comprising the amino acid sequence of SEQ ID NO. 4 with 1 or more amino acids deleted, substituted, or added, the polypeptide having at least 50% residual thioredoxin reductase activity after heat treatment at 100° C. for 0.5 hour.

18. DNA of (8-1), (8-2) or (8-3) below:
(8-1) DNA comprising the base sequence of SEQ ID NO. 3;
(8-2) DNA comprising DNA which hybridizes under stringent conditions with DNA consisting of the base sequence of SEQ ID NO. 3 and encodes a polypeptide having at least 50% residual thioredoxin reductase activity after heat treatment at 100° C. for 0.5 hour; and
(8-3) DNA encoding the polypeptide according to claim 17

19. A vector comprising the DNA according to claim 18.

20. A transformant comprising the vector according to claim 19.

21. A method for producing a heat resistant thioredoxin reductase, comprising the steps of culturing the transformant of claim 20, and collecting the heat resistant thioredoxin reductase from the transformant.

22. A heat resistant thioredoxin reductase according to claim 15 whose activity is substantially unimpaired by heat treatment at 100° C. for 0.5 hour.

23. A heat resistant thioredoxin reductase according to claim 22, derived from hyperthermophilic archaea Pyrococcus horikoshii.

24. A polypeptide of (4-1) or (4-2) below:
(4-1) a polypeptide comprising the amino acid sequence of SEQ ID NO. 10; and
(4-2) a polypeptide comprising the amino acid sequence of SEQ ID NO. 10 with 1 or more amino acids deleted, substituted, or added, the polypeptide having thioredoxin reductase activity which is substantially unimpaired by heat treatment at 100° C. for 0.5 hour.

25. DNA of (9-1), (9-2) or (9-3) below:
(9-1) DNA comprising the base sequence of SEQ ID NO. 9;
(9-2) DNA comprising DNA which hybridizes under stringent conditions with DNA consisting of the base sequence of SEQ ID NO. 9 and encodes a polypeptide whose thioredoxin reductase activity is substantially unimpaired by heat treatment at 100° C. for 0.5 hour; and
(9-3) DNA encoding the polypeptide according to claim 24.

26. A vector comprising the DNA of claim 25.

27. A transformant comprising the vector of claim 26.

28. A method for producing a heat resistant thioredoxin reductase, comprising the steps of culturing the transformant of claim 27 and collecting the heat resistant thioredoxin reductase from the transformant.

29. A heat resistant thioredoxin peroxydase having at least 50% residual activity after heat treatment at 100° C. for 0.5 hour.

30. A heat resistant thioredoxin peroxydase according to claim 29, derived from hyperthermophilic archaea Aeropyrum pernix.

31. A polypeptide of (5-1) or (5-2) below:
(5-1) a polypeptide comprising the amino acid sequence of SEQ ID NO. 6; and
(5-2) a polypeptide comprising the amino acid sequence of SEQ ID NO. 6 with 1 or more amino acids deleted, substituted, or added, the polypeptide having at least 50% residual thioredoxin peroxydase activity after heat treatment at 100° C. for 0.5 hour.

32. DNA of (10-1), (10-2) or (10-3) below:
(10-1) DNA comprising the base sequence of SEQ ID NO. 5;
(10-2) DNA comprising DNA which hybridizes under stringent conditions with DNA consisting of the base sequence of SEQ ID NO. 5 and encodes a polypeptide having at least 50% residual thioredoxin peroxydase activity after heat treatment at 100° C. for 0.5 hour; and
(10-3) DNA encoding the polypeptide according to claim 31

33. A vector comprising the DNA according to claim 32.

34. A transformant comprising the vector according to claim 33.

35. A method for producing a heat resistant thioredoxin peroxydase, comprising the steps of culturing the transformant of claim 34, and collecting the heat resistant thioredoxin peroxydase from the transformant.

36. A method for purifying a heat resistant protein, comprising a heating step in which a solution of the heat resistant protein to be purified is incubated for 10 to 120 minutes at a temperature such that incubating the protein for 10 to 30 minutes results in at least 60% residual activity and that is at least 10° C. higher than the critical growth temperature of the host producing the protein.
